(19) United States
(12) Patent Application Publication
(10) Pub. No.: US 2023/0226237 A1
(43) Pub. Date: Jul. 20, 2023

(54) IMPROVEMENTS IN OR RELATING TO DISINFECTION OF OBJECTS

(71) Applicant: HAI CLEAN LIMITED, Dumbarton (GB)

(72) Inventor: David TONERY, London (GB)

(73) Assignee: HAI CLEAN LIMITED, Dumbarton (GB)

(21) Appl. No.: 18/016,421

(22) PCT Filed: Jul. 19, 2021
(86) PCT No.: PCT/GB2021/051852

§ 371 Date: Jan. 16, 2023

(30) Foreign Application Priority Data
Jul. 17, 2020 (GB) 2011056.5

(51) Int. Cl. A61L 2/24; A61L 2/10; A61L 2/26

(52) U.S. Cl. CPC A61L 2/24; A61L 2/10; A61L 2/26; A61L 2202/122; A61L 2202/14; A61L 2202/11

(57) ABSTRACT

A disinfection apparatus for disinfecting an object is disclosed. The disinfection apparatus comprises at least one disinfection unit operable to perform a disinfection operation on the object to be disinfected, an outer casing and at least one actuating door. The outer casing and the at least one actuating door define a disinfection compartment. The apparatus includes at least one position sensor for detecting the presence of an object to be disinfected having been positioned within the disinfection compartment, at least one obstruction sensor operable to detect whether at least a first safety zone is clear, and a central control unit, the central control unit being operable to control operation of the at least one actuating door, the at least one position sensor, the at least one obstruction sensor, and the at least one disinfection unit.

### FIELD OF THE INVENTION

This invention relates to disinfection of objects and particularly, but not exclusively, to apparatuses and methods for disinfection of shopping trolleys or carts.

### BACKGROUND TO THE INVENTION

During pandemics, there is a need to limit or prevent the spread of infection between people. This is particularly true for spaces or situations where large numbers of people are able to interact with objects, or wherein objects may be otherwise exposed to potential infection risks. It is well understood that such objects may increase the spread of infection. Accordingly, there is a need to disinfect such objects to ensure that the risk of the objects aiding in the spread of an infection is mitigated.

Typically, disinfection of an object is inconvenient and time consuming for the user or persons involved. Additionally, in some instances, the disinfection operation itself may act as an infection risk to the user, since known disinfection apparatuses and solutions require the user to interact with the apparatus or solution.

The inventor has appreciated the problems with existing solutions.

### SUMMARY OF THE INVENTION

In accordance with a first aspect of the invention, there is provided a disinfection apparatus for disinfecting an object, the disinfection apparatus comprising:
at least one disinfection unit operable to perform a disinfection operation on the object to be disinfected;
an outer casing;
at least one actuating door, the outer casing and the at least one actuating door defining a disinfection compartment;
at least one position sensor for detecting the presence of an object to be disinfected having been positioned within the disinfection compartment;
at least one obstruction sensor operable to detect whether at least a first safety zone is clear; and
a central control unit, the central control unit being operable to control operation of the at least one actuating door; the at least one position sensor; the at least one obstruction sensor; and the at least one disinfection unit.

The disinfection apparatus may be adapted to disinfect any suitable object, product, assembly of objects or products or combination of objects or products. The outer casing of the disinfection apparatus may be adapted or shaped to substantially contain any suitable object, product, assembly of objects or products or combination of objects or products.

The outer casing of the disinfection apparatus may have any suitable outside shape or configuration.

The outer casing of the disinfection apparatus may have any suitable inside shape or configuration thereby to form a disinfection compartment. During use, an object to be disinfected is positioned inside the disinfection compartment.

The outer casing of the disinfection apparatus may be comprised of a suitable material. The outer casing may be comprised of a metallic material. The outer casing may be comprised of a metal or a metallic alloy. The outer casing may be comprised of a plastic material. The outer casing may be made of natural materials, e.g., (without limitation) wood or bamboo. The outer casing may be made of a glass material. The outer casing may be comprised of a plurality of materials. The outer casing may be comprised of a plurality of layers of materials. The outer casing may be comprised of a combination of materials.

At least some of the surfaces of the disinfection compartment may be substantially covered with a reflective surface element, including (without limitation): polished stainless steel; reflective paint; or reflective aluminium foil. The reflective surface element may include reflective foil, plastic foil, or an impregnated plastic foil.

The disinfection apparatus may comprise one or more actuating doors. The one or more doors allow access to the disinfection compartment, and may allow the disinfection compartment to be closed and/or sealed during the disinfection process. Given that disinfection processes may involve procedures that are potentially unsafe for the user, other persons or animals (e.g., pets or other animals), closing and sealing the disinfection compartment during operation thereof prevents or reduces the risk of harm to the user, other persons or animals.

The one or more actuating doors may be positioned or located in a suitable location of the disinfection apparatus. One or more of the at least one actuating doors may be located at a first region of the disinfection apparatus and one or more of the at least one actuating doors may be located at a second region of the disinfection apparatus. The first region may be located at an end region of the outer casing. The first region may be an entrance to the disinfection unit. The second region may be an exit of the disinfection unit. The first and second region may be arranged at opposing ends of the outer casing. In some examples, the disinfection apparatus may have actuating doors located at opposing ends of the outer casing.

The one or more actuating doors may be arranged or formed to actuate in a suitable manner. The one or more actuating doors may comprise an attachment mechanism for attaching the one or more actuating door to the outer casing. The attachment mechanism may include one or more hinges, or the like. The one or more actuating doors may comprise a number of sections and may be formed as roller-type doors. The one or more actuating doors may be formed of a flexible material and may comprise a roller type mechanism. The one or more actuating doors may comprise one or more rotary doors.

The one or more actuating doors may comprise additional features that improve the functionality thereof. At least one of the one or more actuating doors may comprise a cover member configured to reduce or prevent the transmission of electromagnetic radiation therethrough. The electromagnetic radiation may be ultraviolet light, visible light and/or infrared light. The cover member may be a light-trap, an absorber member, or the like. The cover member may be arranged at an edge portion of at least one of the one or more actuating doors. The edge portion may be a bottom edge of the one or more actuating doors. The cover member thereby allows an object to be disinfected to be moved in and out of the apparatus but reduces or prevents any light propagating into or out of the outer casing when the one or more actuating doors are closed. Each of the one or more actuating doors may comprise one or more cover members.

At least one of the one or more actuating doors may be attached to a suitable door actuating unit comprising a suitable door actuating mechanism. At least one of the door actuating units may comprise a powered or power-assisted door actuating mechanism. The at least one door actuating unit may be connected directly or indirectly with the actuating mechanism of the one or more actuating door.

At least one of the or more actuating doors may comprise a door control unit that controls operation of the at least one actuating door.

At least one of the one or more actuating doors may comprise one or more additional features. In some examples, the at least one actuating door comprises a buffer element. The buffer element may be operable to protect the actuating door. The buffer element may be operable to at least partially aid in correctly positioning the object to be disinfected within the disinfection compartment. In some examples, the buffer element is comprised of a resilient material. The resilient material may include a foam material. The resilient material aids positioning of the object to be disinfected within the disinfection compartment by allowing the object to be disinfected to rebound from the buffer element when making contact therewith.

The disinfection apparatus may comprise one or more locking mechanisms operable to lock the, or each, actuating door. The locking mechanism(s) may be controlled by a central control unit.

Each actuating door may comprise a door control unit that controls operation of the respective actuating door. The door control unit may be connected to one or more of: a respective door actuation mechanism operable to actuate the respective door; a respective locking mechanism operable to lock the respective actuating door; and the central control unit. In some examples, the door control unit may be connected to one or more sensors (which will be described in more detail in the following) in addition to or alternatively to being connected to the central control unit.

The door control unit may be operable to cause the one or more actuating doors to be actuated in response to a triggering event. Triggering events include, without limitation: the disinfecting apparatus being in a standby state and an object to be disinfected being detected in the first safety zone; a disinfecting operation being completed; or an emergency trigger being operated. In any example, on occurrence of a triggering event, the door control unit may be operable to send a signal to the respective door actuation mechanism, which in turn actuates the actuating door.

Each of the one or more actuating doors may be movable between an open position and a closed position.

Each of the one or more actuating doors may additionally comprise one or more door sensors. The one or more door sensors may be connected to the door control unit. In some examples, the one or more door sensors comprise a pressure sensor. The pressure sensor may be operable to detect whether the actuating door hits an obstruction during movement. If this happens, the door control unit or central control unit may be operable to switch the actuating door into the open position. The one or more door sensors may comprise a limit switch operable to detect if the, or each, actuating door is in an open position or in a closed position.

The door control unit may be a separate unit. The door control unit may be comprised in the central control unit of the disinfection apparatus.

The disinfection apparatus may comprise one or more additional internal or external features.

The disinfection apparatus may comprise one or more access members. The access member(s) may include ramp(s), inclined portion(s), support member(s), or the like. The, or each, access member may be positioned adjacent to the one or more actuating doors. The one or more access members may facilitate access to the disinfection apparatus, in particular for objects that are either heavy or have wheels (such as, without limitation, shopping trolleys, carts or the like). The access member may comprise a surface cover configured to mitigate or prevent accidental slipping of the object to be disinfected or persons on the access member. The surface cover may be made of a rubber material. The surface cover may comprise safety features operable to prevent objects or persons slipping.

The disinfection apparatus may comprise one or more guide members. The, or each guide member may be located external to the outer casing. The one or more guide members may include one or more barriers, guard members, rail members, or guard rails, or the like. The guide member(s) may be positioned adjacent or proximal to at least one of the one or more actuating doors. The guide members rails may be formed, arranged and/or positioned so as to facilitate the movement of objects to be disinfected into and out of the disinfection apparatus. The guide members may be arranged to facilitate alignment of the object to be disinfected with the actuating doors. The guide member(s) may be formed, arranged and/or positioned so as to prevent undesired or accidental movement by the object to be disinfected in proximity to the disinfection apparatus during disinfection operations.

The disinfection apparatus may comprise one or more disinfection units. The disinfection units are operable to disinfect the object to be disinfected in a suitable manner. Each disinfection unit may be provided with one or more disinfection elements suitable for disinfecting the object to be disinfected. In some examples, the one or more disinfection elements are operable to provide one or more oxidising compounds or materials to the object to be disinfected. The oxidising compounds or materials may be used to destroy microbes by causing oxidative damage to the cell membrane and other sensitive structures. In some examples, the one or more disinfection elements may comprise means for providing or generating ozone, plasma or corona discharges.

In some examples, one or more of the disinfection elements may comprise one or more radiation sources that generate or emit electromagnetic radiation. The electromagnetic radiation may comprise radiation within one or more wavelength ranges, including (but not limited to): ultraviolet (UV) wavelengths; visible wavelengths; or infrared radiation.

In some examples, the one or more radiation sources may be a pulsed ultraviolet light source, which is operable to deliver pulsed, high-intensity UV radiation. In some examples, the one or more radiation sources may be operable to deliver high intensity narrow-spectrum radiation. In an example, the high intensity narrow-spectrum radiation comprises visible light with a centre wavelength of substantially 405 nm. Exposure to light of this wavelength induces a photo-excitation reaction within microorganisms which results in the production of reactive oxygen species, and oxidative damage to the microbial cell. Such lethal damage to microbial cells can be exerted at radiation levels or doses that are safe for human exposure.

In some examples, the one or more disinfection elements are operable to generate and provide an electric field to the object to be disinfected. The electric field may be a pulsed electric field. Exposure of microbes to very short and high voltage pulses causes electroporation (bursting) of the microbe's cell membrane.

The disinfection apparatus may comprise any suitable number of disinfection units. In some examples, the disinfection apparatus may comprise (without limitation): at least two disinfection units; at least three disinfection units; at least four disinfection units; at least five disinfection units; at least six disinfection units; at least seven disinfection units; at least eight disinfection units; at least nine disinfection units; or at least ten disinfection units. In an example, the disinfection apparatus may comprise four disinfection units.

The radiation sources may be positioned, formed or arranged so as to illuminate or irradiate an object positioned within the disinfection compartment.

The radiation sources may be formed or arranged to emit substantially directional radiation. This enables the amount of radiation used for the disinfection process to be maximised, thereby increasing disinfection efficacy. In some examples, the radiation sources comprise directional radiation sources. In other examples, the radiation sources comprise omnidirectional radiation sources. In such examples, the radiation sources may comprise one or more optical elements arranged or formed to direct radiation in a plurality of directions. In some examples, the optical units may be arranged or formed to direct radiation in a first direction and at least a second direction. In some examples, the optical units may be arranged or formed to direct radiation in a first direction and at least a second direction.

The one or more radiation sources may be arranged so as to illuminate or irradiate substantially all outer surfaces of the object to be disinfected. At least one of the one or more radiation sources may be positioned so as to substantially illuminate the lower surfaces of the object to be disinfected. At least one of the one or more radiation sources may be positioned so as to substantially illuminate the upper surfaces of the object to be disinfected. At least one or the one or more radiation sources may be positioned so as to substantially illuminate the front surfaces of the object to be disinfected. At least one or the one or more radiation sources may be positioned so as to substantially illuminate the rear surfaces of the object to be disinfected. At least one or the one or more radiation sources may be positioned so as to substantially illuminate the side surfaces of the object to be disinfected.

The one or more disinfection units may be arranged in a fixed location relative to the outer casing. The one or more disinfection units may be configured to be movable relative to the outer casing. The one or more disinfection units may be configured to substantially orbit the object to be disinfected. The one or more radiation sources may be arranged in fixed locations or positions. The one or more radiation sources may be arranged to be movable. In some examples, the one or more radiation sources may be arranged to substantially orbit the object to be disinfected.

The disinfection apparatus may comprise one or more positioning aids operable to facilitate the correction positioning of the object to be disinfected. The positioning aids may have any suitable or relevant shape or form to receive at least a portion of the object to be disinfected. The positioning aid(s) may be located at least partially within the outer casing.

In some examples, the positioning aids may comprise at least one indent ridge. The at least one indent ridge may be positioned in an inner bottom surface or floor of the disinfection apparatus. In other examples, the positioning aids may comprise at least one indent ridge positioned in an inner side surface of the disinfection apparatus. In some examples, the positioning aids may comprise at least one indent ridge positioned in an inner top surface of the disinfection apparatus.

The positioning aids may be adapted to receive at least a portion of the object to be disinfected. The positioning aid(s) may be adapted to receive one or more wheels of a n object to be disinfected, such as the wheels of a shopping trolley. The positioning aids may comprise one or more indent ridges corresponding to the number of wheels of an object to be disinfected, such as a shopping trolley or the like, the indent ridges being positioned in an inner bottom surface or floor of the disinfection apparatus. The indent ridges may be positioned relative to each other in a configuration that substantially corresponds to that of the wheel configuration of a shopping trolley.

The positioning aids may comprise a plurality of actuators operable to facilitate movement of the object to be disinfected (e.g., a shopping trolley) into and out of the disinfection compartment. The plurality of actuators may be operable to move the object to be disinfected into and out of a first location. The actuators may be operable to maintain the object to be disinfected in the first location during disinfection operations. The actuators may comprise rollers or the like.

The disinfection apparatus may comprise one or more positioning sensors operable to detect the position of an object to be disinfected inside the disinfection compartment. The one or more positioning sensor may be operable to determine that an object to be disinfected is in the correct position inside the disinfection compartment.

The one or more positioning sensors may be of any suitable type and may detect the position of an object to be disinfected. The one or more positioning sensors may be (without limitation): electromagnetic sensors operable to detect a suitable type of electromagnetic radiation, e.g., infrared, visible or ultraviolet radiation; pressure sensors; or audio sensors. In some examples, the positioning sensors may be (without limitation) one or more of: a laser displacement sensors; a microwave sensor; an inductive proximity sensor; a pressure or load cell sensor; a 2-D or 3-D vision sensor; a laser positioning sensor, a photoelectric sensor; or a fibre optic sensor.

In some examples, wherein the positioning aids comprise one or more indent ridges, the one or more positioning sensors may be positioned substantially in or adjacent to the one or more indent ridges. In an example, the positioning aids comprises a plurality of positioning sensors corresponding to the plurality of indent ridges. In a specific example, each positioning sensor may be positioned substantially within, and optionally centred on, a respective indent ridge. In a specific example, each positioning sensor is a pressure sensor.

In an example, the one or more position sensors may be a proximity sensor that detects the proximity of an object. In an example, the one or more positioning sensors may be electromagnetic sensors adapted and positioned to detect when an object to be disinfected is located in the disinfection compartment. The one or more electromagnetic positioning sensors may use any suitable radiation, including (but not limited to): ultraviolet (UV) radiation; visible radiation; infrared radiation; microwave radiation; or radio frequency radiation. The one or more positioning sensors may be positioned on a suitable interior surface of the disinfection compartment. In a specific example, the one or more positioning sensors is a photoelectric sensor positioned on one of the one or more actuating doors of the disinfection apparatus. The central control unit may be operable to obtain a status of the position of the object in the disinfection compartment. The central control unit may be operable to indicate the status of the position of the object in the disinfection compartment.

The disinfection apparatus may comprise a control equipment box, the control equipment box comprising the central control unit operable to control the operation of the disinfection apparatus.

The central control unit may comprise a processing unit and a memory unit. The central control unit may comprise a display unit and a user input unit. The central control unit may be connectable with a display unit and/or a user input unit. The central control unit may comprise an audio speaker unit for emitting warnings, operation statuses or instructions to a user.

The central control unit may comprise a communication unit operable to enable the central connection unit to connect to and communicate with other relevant units or devices in a suitable manner. In some examples, the communication unit is operable to connect with a communications network (e.g., a 4G or 5G network). In some examples, the communication unit is operable to connect with a data network by way of a wireless connection or a wired connection (e.g., WiFi, ethernet, ADSL or a fibre connection). In some examples, the communication unit is operable to connect directly with one or more external devices (e.g., Bluetooth, NFC or other short-range communication protocols). The communication unit may be operable to communicate with any relevant devices under any suitable circumstances (e.g., communicating with a repair centre in the event of a fault in the operation of the disinfection apparatus).

The central control unit may be operable to carry out any relevant operations. The central control unit may carry out monitoring and diagnostics of the operation of the disinfection apparatus to ensure that all components are functioning correctly.

The central control unit may monitor the number of times that the disinfection device has been used, to determine when the apparatus should be checked or maintained.

The disinfection apparatus may comprise one or more obstruction sensors operable to detect whether the one or more safety zones are clear or unobstructed. The obstruction sensors ensure that the disinfection operation is only carried out when the user, or other objects that are not to be disinfected, is in a safe location. Given that disinfection may be harmful to the user, or other persons (such as children), it is important that the disinfection apparatus is not able to carry out disinfection operations whilst the user or other persons are in a location in which they may be harmed by the disinfection operation.

The obstruction sensors may comprise one or more internal obstruction sensors operable to detect whether a person or animal (e.g., a pet) is inside the disinfection compartment. Such sensors may indicate whether any persons, animals or other objects are located inside the disinfection compartment, thereby to prevent accidental or premature commencement of the disinfection operation.

The one or more internal obstruction sensors may be of any suitable type and may use any suitable detection mechanism. The one or more internal obstruction sensors may be an electromagnetic sensor using radiation having a suitable wavelength range. The one or more internal obstruction sensors may, without limitation, use one or more of: infrared radiation; substantially visible radiation; or ultraviolet radiation. The one or more internal obstruction sensors may be a camera. The one or more internal obstruction sensors may comprise a radiation source and a radiation detector operable to detect radiation emitted by the radiation source. The one or more internal obstruction sensors may be a photoelectric cell comprising a radiation source and a reflector. The one or more internal obstruction sensors may be a motion sensor. The motion sensor may be a microwave doppler motion sensor.

In an example, the one or more internal obstruction sensors may be operable to detect radiation within a radio frequency spectrum, such as (without limitation) Bluetooth, WiFi or NFC. In such an example, the one or more internal obstruction sensors may be used to detect the presence of a mobile user device, which would typically indicate the presence of a user within the disinfection compartment.

In some examples, the one or more internal obstruction sensors comprise a plurality of sensors of at least one type. The one or more internal obstructions sensors may comprise a plurality of sensors of at least two different types. By using a plurality of sensors, preferably of at least two different types, safety of operation may be increased by ensuring that several different types of sensing is performed. This increases the safety of operation of the device.

The obstruction sensors may comprise one or more external obstruction sensors operable to detect the presence of an object or person and/or movement in one or more areas outside the disinfection compartment or surrounding the outer casing of the disinfection apparatus.

The one or more external obstruction sensors may be of any suitable type and may use any suitable detection mechanism. At least one of the external obstruction sensors may be a pressure sensor. At least one of the external obstruction sensors may be a sensor for detecting electromagnetic radiation, including (but not limited to): a photocell operable to detect substantially visible radiation; an infrared sensor; or an ultraviolet sensor. At least one of the external obstruction sensors may be an audio sensor. The at least one external obstruction sensors may (without limitation) be: a laser displacement sensors; a microwave sensor; an inductive proximity sensor; a pressure or load cell sensor; a 2-D or 3-D vision sensor; a laser positioning sensor; a photoelectric sensor; or a fibre optic sensor.

The one or more external obstruction sensors may be positioned in any suitable location or locations. The one or more external obstruction sensors may be positioned on or attached to the outer casing. The one or more external obstruction sensors may be positioned on or attached to the one or more actuating doors. The one or more external obstruction sensors may be positioned on or attached to the one or more additional features, including (but not limited to) the one or more access members; one or more of the outer surfaces of the outer casing; one or more of the actuating doors; or the one or more guide members.

In some examples, the one or more external obstruction sensors may comprise a pressure sensor located in the one or more access members, the pressure sensor being operable to detect the presence of an object or a person on the access member. In some examples, the one or more external obstruction sensors may comprise a photocell positioned in the one or more guide members, the photocell being operable to detect the presence of an object or person located substantially adjacent to or between the guide members.

The disinfection apparatus may comprise one or more indicator units operable to indicate operational status of the disinfection apparatus to a user. This enables a user to receive an indication of the status of the apparatus itself (e.g., whether the apparatus is ready to receive an object to be disinfected) and/or an indication of the progress of the disinfection operation.

The one or more indicator units may comprise one or more visual indicators, e.g., lighting units or visual display units. The one or more indicator units may comprise audio indicators, e.g., speakers or other emitters. The one or more indicator units may comprise both one or more visual indicators and one or more audio indicators. The one or more indicator units may comprise haptic feedback indicators.

The one or more indicator units may be operable to indicate the status of the position of the object in the disinfection compartment. The status may be indicated with a traffic-light system, a green visual indicator, a red visual indicator, or any suitable indicator method to display the status.

The disinfection apparatus may comprise a monitoring system, the monitoring system being operable to display to a user the disinfection compartment during disinfection operations. This allows the user to monitor the progress of the disinfection operation. The monitoring system may comprise a detector positioned inside the disinfection compartment. The detector may comprise an imaging sensor, such as a camera, a closed circuit TV camera or other video monitoring unit.

The monitoring system may comprise one or more illumination sources operable to illuminate at least a portion of the object to be disinfected. The one or more illumination sources may be operable to illuminate at least a portion of the object to be disinfected when the object is located in the disinfection unit. The one or more illumination sources may be UV light sources, UVA light sources, or the like. The monitoring system may be operable to capture one or more images of the disinfection unit when illuminated. The central control unit may be operable to save the captured images. The central control unit may be operable to send the, or each captured image to a computing device, to a distributed computing network, or to a wireless communications network. The central control unit may be operable to activate the one or more illumination sources of the monitoring system before a disinfection operation begins and/or after the disinfection operation is complete. The monitoring system may be configured to detect the presence of pathogens, bacteria or viruses. The detection of pathogens may be carried out using fluorescence imaging, or any suitable imaging method of detecting or inferring the presence of pathogens, bacteria or viruses.

The monitoring system may comprise a display unit. The display unit may be positioned on the outer casing of the disinfection apparatus. The display unit may be connected to the detector, thereby to allow the user to observe the inside of the disinfection apparatus. In some examples, the monitoring system is connected to the display unit of the central control unit. In some examples, the monitoring system may be operable to connect to a remote device, e.g., a user device, by way of a communication unit. This would enable a user or other person to monitor the operation of the apparatus remotely. The monitoring system may comprise one or more display units, or a plurality of display units. A display unit may be located on or adjacent to the at least one actuating door. A display unit may be located on or adjacent to at least one guide member.

The central control unit may be operable to obtain a status of the at least one actuating door, the at least one position sensor, the at least one obstruction sensor, and/or the at least one disinfection unit. The status may include an indication of whether the component is in compliance with one or more pre-defined operating parameters. The status may include the status of one or more of the components of the at least one actuating door, the at least one position sensor, the at least one obstruction sensor, and/or the at least one disinfection unit. The disinfection apparatus may comprise one or more condition monitoring devices operable to provide the status of the at least one actuating door, the at least one position sensor, the at least one obstruction sensor, and/or the at least one disinfection unit.

The status may include one or more fault conditions. The one or more fault conditions may be associated with the at least one actuating door, the at least one position sensor, the at least one obstruction sensor, and/or the at least one disinfection unit. The one or more fault conditions may be associated with one or more of the components of the at least one actuating door, the at least one position sensor, the at least one obstruction sensor, and/or the at least one disinfection unit.

One or more of the condition monitoring device(s) may be configured to provide a status of the one or more disinfection elements. One or more of the condition monitoring device(s) may be configured to measure a parameter associated with the operation of the one or more disinfection elements.

The disinfection unit may comprise one or more backup disinfection elements. The central control unit may be operable to activate one or more backup disinfection elements in response to the status of the one or more disinfection elements being out of compliance with a predetermined value.

The one or more indicator units may be configured to indicate the status of the at least one actuating door, the at least one position sensor, the at least one obstruction sensor, and/or the at least one disinfection unit. The one or more indicator units may be configured to indicate the status of the one or more disinfection elements. The one or more indicator units may be configured to indicate the status of the one or more backup disinfection elements.

The central control unit may be operable to transmit one or more signals indicative of the status of the at least one actuating door, the at least one position sensor, the at least one obstruction sensor, and/or the at least one disinfection unit, to a computing device, a distributed computer network, and/or a computing device connected to a distributed computer network. The computing device may be a PC, laptop, or smart phone, or the like.

The central control unit may be operable to indicate one or more recommended scheduled maintenance periods, such as a service date, or the like. The one or more indicator units may be operable to indicate the one or more recommended scheduled maintenance periods.

The central control unit may be operable to store operation data associated with the use of the disinfection apparatus. The operation data may include the number of times the disinfection apparatus was used. A single use of the apparatus may be defined in any suitable way, such as by one or more disinfection operations being carried out. The operation data may include the number of objects that have been disinfected using the disinfection apparatus. The operation data may include time data associated with how long the disinfection apparatus has been used for. The time data may include the time at which the disinfection apparatus was used at and/or the date on which it was used.

The one or more indicator units may be operable to indicate at least some of the operation data. The one or more indicator units may be operable to indicate at least some of the time data. The one or more indicator units may be operable to indicate the number of times the disinfection apparatus is used, and/or the number of objects that have been disinfected using the disinfection apparatus.

The central control unit may comprise an object communication element. The object communication element may be operable to communicate with one or more identifier elements associated with the object to be disinfected. The one or more identifier elements may be located on, or in, the object to be disinfected. The one or more identifier elements may be transmitter elements, receiver elements, transceiver elements, tags, RFID tags, internet-of-things (IoT) devices, or the like.

The object communication element may be operable to communicate with the one or more identifier elements using one or more communication protocols. The communication protocol may be a wireless communication protocol, optionally WiFi.

The object communication element may be operable to obtain object data from the one or more identifier elements. The object data may include data on the type of object, the identity of the object (e.g. a unique identifier or code allocated to the object), information on when the object was disinfected by a disinfection apparatus, and/or location data associated with the object.

The central control unit may be operable to communicate object data to one or more computing devices, optionally over a distributed computing network or wireless communication link.

The central control unit may be operable to display at least some of the object data. The one or more indicator units may be operable to indicate at least some of the object data.

The disinfection apparatus may comprise an object sensor operable to detect at least one property of the object to be disinfected. The object sensor may be operable to detect at least one property of the object to be disinfected when the object is located within the disinfection compartment. The at least one property may include the type of object. The object sensor may be operable to determine the shape of at least a portion of the object to the disinfected. The central control unit may be operable to classify the object according to the at least one property detected by the object sensor. The central control unit may be operable to classify the object according to the shape detected by the object sensor. The central control unit may be operable to classify the object as a simple shape object or a complex shape object. The disinfection apparatus may comprise one or more object sensors. The disinfection apparatus may comprise an array of object sensors.

The, or each, object sensor may be an optical sensor, a camera, electro-optical sensor, infra-red sensor, or the like. The object sensors may be arranged in a grid pattern.

The central control unit may be operable to adjust the operating parameters of the at least one disinfection unit based, at least in part, on data received from the object sensor. The central control unit may be operable to adjust the operating parameters of the one or more disinfection elements based, at least in part, on data received from the object sensor. The central control unit may be operable to adjust the operating parameters of the at least one disinfection unit based, at least in part, on the classification of the object. The central control unit may be operable to adjust the operating parameters of the one or more disinfection elements based, at least in part, on the classification of the object.

The monitoring system may be operable to record one or more images and/or one or more videos of at least a portion of the disinfection compartment during disinfection operations.

The monitoring system may be operable to detect shadows on the object to be disinfected. The monitoring system may be operable to detect one or more shadows on the object to be disinfected during disinfection operations. The one or more shadows may be detected when the one or more disinfection elements are active. The central control unit may be operable to modify the operation of the one or more disinfection elements based on the detection of one or more shadows on the object to be disinfected. Modifying the operation of the one or more disinfection elements may include activating or deactivating some or all of the disinfection elements, and/or modifying the intensity of some or all of the disinfection elements.

The one or more disinfection elements may be located at or adjacent to the inner top surface of the disinfection unit, the inner bottom surface of the disinfection unit and/or at least one of the inner side surfaces of the disinfection unit. Modifying the operation of the one or more disinfection elements may include activating or deactivating one or more disinfection elements located at or adjacent to the inner top surface of the disinfection unit, the inner bottom surface of the disinfection unit and/or at least one of the inner side surfaces of the disinfection unit. Modifying the operation of the one or more disinfection elements may include modifying the intensity of one or more disinfection elements located at or adjacent to the inner top surface of the disinfection unit, the inner bottom surface of the disinfection unit and/or at least one of the inner side surfaces of the disinfection unit.

One or more of the disinfection elements may be movable between at least two positions in the disinfection unit. One or more of the disinfection elements may be movable disinfection elements. One or more of the disinfection elements may be mounted to an actuator device configured to move the disinfection element between the at least two positions. The actuator device may be a vehicle, a robotic vehicle, or the like. The central control unit may be operable to control the operation of the actuator device. Modifying the operation of the one or more disinfection elements may include moving one or more disinfection elements between the at least two positions. The at least two positions may include a first, stowed position, and a second, deployed position. The central control unit may be operable to move at least one disinfection element from the first, stowed position to the second, deployed position prior to, or during, the disinfection operation. The central control unit may be operable to move the at least one disinfection unit from the second, deployed position, to the first, stowed position, once the disinfection operation has been completed, or during the disinfection operation.

The user input unit may be operable to move one or more of the disinfection elements between the at least two positions.

The central control unit may be operable to trigger one or more alarms based on data received from the one or more external obstruction sensors. The one or more alarms may include visual alarms or warnings and/or audio alarms or warnings. The, or each alarm may be displayed on the display unit.

The central control unit may be operable to prompt a person to use the disinfection apparatus. The central control unit may be operable to prompt a person to use the disinfection apparatus based on data received from the one or more external obstruction sensors. The prompt may include a visual prompt and/or audio prompt. The, or each, prompt may be displayed on the display unit.

The external obstruction sensor may be operable to measure or infer a person's body temperature. The central control unit may be operable to trigger a warning or alarm if a person's body temperature is above a predetermined threshold and/or below a predetermined threshold.

The central control unit may be configured to automatically move one or more of the at least one actuating doors from a closed position to an open position. The central control unit may be configured to automatically move one or more of the at least one actuating doors from a closed position to an open position in response to the detection of a person outside of the disinfection apparatus. The central control unit may be configured to automatically move one or more of the at least one actuating doors from a closed position to an open position in response to the detection of a person outside of the disinfection apparatus by the one or more external obstruction sensors.

The central control unit may be operable to move one or more of the at least one actuating doors from the closed position to the open position when one or more of the external obstruction sensors detects the presence of a user. The central control unit may be operable to move the at least one actuating doors from the open position to the closed position when the object to be disinfected is in the first safety zone. The central control unit may be operable to move one or more of the at least one actuating doors from the closed position to the open position when the one or more disinfection operations are complete.

The central control unit may be configured to use data from the one or more external obstruction sensors to classify the person detected by the one or more external obstruction sensors. For example, the person may be classified by height, weight, or any suitable parameter. The classification may include classifying the person as an adult or child, or an animal. The central control unit may be operable to allocate user permission to one or more classifications of a person and to deny user permission to one or more classifications of a person. The central control unit may be operable to maintain the at least one actuating door in the closed position, if a person who does not have user permission is detected by the one or more external obstruction sensor. The central control unit may be operable to allow the operation of the disinfection apparatus when a user who has user permission is detected by the one or more external obstruction sensors.

The disinfection apparatus may comprise one or more first external obstruction sensors, orientated towards a first sensing area, and one or more second external obstruction sensors, orientated towards a second area. The first sensing area may be at a substantially higher height than the second sensing area. There may be some overlap between the first and second sensing areas, or there may be substantially no overlap. The first sensing area may be defined to detect the presence of a first classification of a person at a predetermined distance range to the first external obstruction sensor. The second sensing area may be defined to detect the presence of a second classification of a person at a predetermined distance range to the second external obstruction sensor.

The second sensing area may be defined to detect the presence of a child at a predetermined distance range to the second external obstruction sensor. The first sensing area may be defined to detect the presence of an adult at a predetermined distance range to the first external obstruction sensor. The first sensing area may be defined such that a child would not be detected at a predetermined distance range to the first external obstruction sensor. In this example, the presence of an adult or child can be determined. It will be understood that the definition of adult and child can be set according to user preference and regulatory requirements. For example, the orientation of the sensors could be used to determine a height threshold between the sensors, or sensor data could be used to infer whether an adult or child is present. The central control unit may be operable to deny user permission unless the first and second external obstruction sensor both detect the presence of a person.

The disinfection apparatus may comprise an air supply system. The air supply system may be operable to provide a pressure difference between the inside of the disinfection unit and the outside of the disinfection unit. The pressure difference may be a positive pressure difference. The air supply system may comprise one or more air inlets and one or more air outlets. The air inlet may be located at an upper region of the disinfection unit. In this arrangement, the pressure difference can mitigate the ingress of debris into the disinfection unit. This arrangement is also thought to assist in disinfecting the disinfection unit.

The disinfection unit may comprise one or more windows. The, or each, window may be configured to filter UV light. The, or each window may include one or more acrylic or plexiglass panes.

The disinfection apparatus may be configured to be movable. The disinfection apparatus may comprise one or more motors configured to move the disinfection apparatus. The disinfection apparatus may comprise one or more wheels. The, or each motor may be operable to move the wheels of the disinfection apparatus. The, or each wheel may be located on a base portion of the outer casing. The, or each motor may include a power supply, which may be a rechargeable power supply.

The disinfection apparatus may be configurable between a stowed state and a deployed state. In the stowed state, the volume of the disinfection unit may be reduced than when in the deployed state. The disinfection apparatus may comprise an extension mechanism operable to configure the disinfection apparatus between the stowed state and the deployed state. The extension mechanism may be configured to extend the disinfection unit vertically and/or horizontally.

In accordance with a second aspect of the invention, there is provided a method for controlling a disinfection apparatus for disinfecting an object to be disinfected, the method comprising:
detecting that the object to be disinfected is positioned in a first location within the disinfection apparatus;
detecting that at least a first safety zone is unobstructed;
closing and locking the disinfection apparatus;
performing one or more disinfection operations;
unlocking and opening the disinfection apparatus; and
resetting the disinfection apparatus when it is detected that object to be disinfected has been removed from the first location.

The method may further comprise detecting that the object to be disinfected is positioned in a second location; and moving an actuating door into an open position to allow access to a disinfection compartment.

The step of detecting that the object to be disinfected is positioned in a first location may comprise using a position sensor to detect that the object to be disinfected is positioned in the first location.

The step of detecting that at least a first safety zone is unobstructed may comprise using one or more obstruction sensors to detect whether the at least one first safety zone is occupied by a person or object. The first safety zone may be located externally to the disinfection device.

The step of detecting that at least a first safety zone is unobstructed may further comprise using one or more obstruction sensors to detect whether a second safety zone is occupied by a person or object, the second safety zone being located inside a disinfection compartment.

In accordance with a third aspect of the invention, there is provided a disinfection apparatus for disinfecting an object, the disinfection apparatus comprising:
at least one disinfection unit operable to perform a disinfection operation on the object to be disinfected;
an outer casing, the outer casing defining a disinfection compartment;
at least one position sensor for detecting the presence of an object to be disinfected having been positioned within the disinfection compartment;
at least one obstruction sensor operable to detect whether at least a first safety zone is clear; and
a central control unit, the central control unit being operable to control operation of the at least one position sensor; the at least one obstruction sensor; and the at least one disinfection unit.

At least a portion of the outer casing may be configurable between an open state and a closed state. In the open state, the object to be disinfected may be locatable in the disinfection compartment.

At least a portion of the outer casing may be movable between the open state and the closed state. At least a portion of the outer casing may be raised or lowered when moving between the open state and the closed state. Moving the at least a portion of the outer casing from the closed state to the open state may result in one or more openings being created in the outer casing for the object to be disinfected to pass through.

Embodiments of the third aspect of the invention may include features of the first and/or second aspects of the invention. Embodiments of the first and/or second aspects of the invention may include features of the third aspect of the invention.

According to a fourth aspect of the invention there is provided a disinfection apparatus for disinfecting an object, the disinfection apparatus comprising:
at least one disinfection unit operable to perform a disinfection operation on the object to be disinfected;
an outer casing, the outer casing defining a disinfection compartment;
at least one position sensor for detecting the presence of an object to be disinfected having been positioned within the disinfection compartment;
a central control unit, the central control unit being operable to control operation of the at least one position sensor and the at least one disinfection unit.

Embodiments of the fourth aspect of the invention may include features of the first second and/or third aspects of the invention. Embodiments of the first, second and/or third aspects of the invention may include features of the fourth aspect of the invention.

### BRIEF DESCRIPTION OF THE DRAWINGS

An embodiment of the invention will now be described, by way of example, with reference to the drawings, in which:

FIGS. 1A-1C show a schematic illustration of an exemplary disinfection apparatus in accordance with the present invention;

FIG. 2 illustrates an exemplary method that may be implemented in a disinfection apparatus;

FIGS. 3A and 3B show an alternative embodiment of the disinfection apparatus in accordance with the present invention; and

FIG. 4 shows another view of the embodiment of FIGS. 3A and 3B.

### DESCRIPTION OF THE PREFERRED EMBODIMENTS

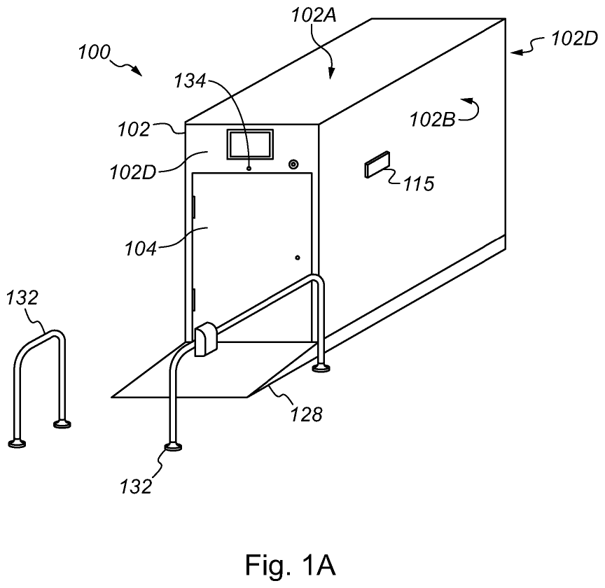

An exemplary embodiment of the present invention will now be described with reference to FIG. 1. It will be appreciated that the embodiment is purely exemplary and that other implementations may easily be envisaged within the scope of the present invention.

A disinfection apparatus 100 comprises an outer casing 102. In the present example, the outer casing is substantially shaped as a cuboid, although it will be appreciated that other shapes may be easily envisaged. The outer casing comprises a top portion 102A and two substantially opposed side portions 102B. The outer casing further comprises a bottom portion 102C. The outer casing further comprises substantially opposed end portions 102D.

The disinfection apparatus has actuating doors 104 at opposing ends of the outer casing. Each of the actuating doors has a door actuating mechanism 106 operable to move the respective actuating door between an open position and a closed position. In the present example, the actuating mechanism is a powered hinge mechanism operable to allow the door to move between the open position and closed position around a substantially vertical axis. However, it will be appreciated that this is purely for exemplary purposes and that other actuating mechanisms could be envisaged. Examples of alternative actuating mechanisms include, but are by no means limited to: roller shutters; concertina doors; a combined door and ramp; sliding doors; or strip curtains.

When in the closed position, the actuating doors 104 and the outer casing 102 form a disinfection compartment 108. In some examples, the disinfection compartment is substantially covered with a reflective surface element, including (without limitation): polished stainless steel; reflective paint; or reflective aluminium foil.

Each of the actuating doors 104 is fitted with a locking mechanism 110 operable to lock the respective door when the disinfection apparatus is in use, thereby to prevent the user or other persons from accessing the inside of the apparatus during disinfection operations. Each locking mechanism is switchable between an unlocked configuration, in which movement of the respective actuating door is enabled, and a locked configuration, in which movement of the respective actuating door is prevented. Further, when in the locked configuration, the locking mechanism ensures that a seal between the respective actuating door and the outer casing is tight in order to prevent ingress or exit of light, compounds or other substances.

In the present example, the locking mechanism 110 is an electromagnetic locking mechanism. The electromagnetic locking mechanism has a door portion, which is positioned on the actuating door and a casing portion that is positioned on the outer casing of the disinfection apparatus. The door portion and the casing portion are both positioned so as to mutually engage to lock the actuating door when the actuating door is in the closed position. It will be appreciated that several specific implementations of such an electromagnetic locking mechanism may be envisaged by the skilled person.

Additionally, the locking mechanism 110 comprises a physical key lock that may be used to secure the respective actuating door during transportation and storage. Each actuating door 104 has a door control unit 112 that controls operation of the respective actuating door. The door control unit is connected to one or more of: the respective door actuation mechanism operable to actuate the respective door; the respective locking mechanism operable to lock the respective actuating door; and a central control unit. In some examples, the door control unit is connected to one or more sensors (which will be described in more detail in the following) in addition to or alternatively to being connected to the central control unit.

The door control unit is operable to cause the one or more actuating doors to be actuated in response to a triggering event. Triggering events include, without limitation: the disinfecting apparatus being in a standby state and an object to be disinfected being detected in the first safety zone; a disinfecting operation being completed; or an emergency trigger being operated. In the present example, on occurrence of a triggering event, the door control unit will send a signal to the respective door actuation mechanism, which in turn actuates the actuating door.

For purposes of the present example, the door control unit 112 has been described as a separate unit, although it will be appreciated that this is purely for explanatory purposes. In the present example, the door control unit is comprised in a central control unit 114 of the disinfection apparatus. It will be appreciated that the skilled person may envisaged alternative implementations within the scope of the present disclosures.

Purely by way of example, in some examples, both actuating doors are controlled by a single door control unit. In some examples, the door control unit is a separate unit from the central control unit of the disinfection apparatus, and may be positioned remotely from the central control unit. In other examples, the one or more door control units are separate from the central control unit but located adjacent or proximal thereto.

The central control 114 unit is positioned in a control equipment box 116 that is located inside the outer casing of the disinfection apparatus. The control equipment box is made of a suitable material. In some examples, the control equipment box is made of a metallic material. The control equipment box is operable to protect the central control unit from being damaged during operation of the disinfection apparatus.

The central control unit 114 comprises a processing unit and a memory unit. The central control unit is connected to a display and user input unit 115. The display and user input unit is operable to display operational status and other relevant information to the user before, during and after operation of the disinfection apparatus. The display and user input unit, in some examples, has an audio speaker unit (not shown) for emitting warnings, operation statuses or instructions to a user. In some examples, the display and user input unit has a payment component (e.g., a NFC reader for accepting card payments) for situations wherein it is intended that a user be charged for operation of the apparatus.

The central control unit 114 comprises a communication unit operable to enable the central connection unit to connect to and communicate with other relevant units or devices in a suitable manner. In some examples, the communication unit comprises means for connecting with a communications network (e.g., a 4G or 5G network). In some examples, the communication unit comprises means for connecting with a data network by way of a wireless connection or a wired connection (e.g., WiFi, ethernet, ADSL or a fibre connection). In some examples, the communication unit comprises means for connecting directly with one or more external devices (e.g., Bluetooth, NFC or other short-range communication protocols). The communication unit is operable to communicate with any relevant devices under any suitable circumstances (e.g., communicating with a repair centre in the event of a fault in the operation of the disinfection apparatus). In some examples, the central control unit has an over-the-air software update capability. In some examples, the central control unit has a physical input software update capability, which enables adjustment of different parameters in the control unit software, including (without limitation) increasing the duration of UV light timing to kill off future virus that may need extra UV light exposure.

The disinfection apparatus comprises one or more disinfection units 118. In the present example, the disinfection apparatus has four disinfection units, each comprising plurality of light sources 120 that emits ultraviolet (UV) radiation. It will be appreciated, however, that other types of radiation could be used alternatively to or in addition to the aforementioned ultraviolet radiation.

The disinfection units 118 are mounted on or attached to a surface of the disinfection compartment 108. As described above, the disinfection compartment is formed of the inside surfaces of the outer casing and the actuating doors. As also described above, in the present example, the disinfection apparatus has four disinfection units. Each disinfection unit extends substantially longitudinally inside the disinfection compartment. In this example, the disinfection units are positioned substantially at each intersection of an inside surface of a side portion 1028 with an inside surface of the top portion 102A or the bottom portion 102C of the outer casing 102. In other terms, each disinfection unit is positioned at the corners of the transverse cross section of the outer casing 102.

The disinfection units 118 are oriented so as to illuminate or irradiate an object placed in the disinfection compartment. In the present example, each of the disinfection units comprises a plurality of UV light sources 120 and a reflective panel 122 operable to reflect any incident radiation substantially towards the object placed in the disinfection compartment. This maximises the amount of UV light directed towards the object to be disinfected, thereby increasing the efficiency and efficacy of the disinfection apparatus. In the present example, each disinfection unit has two subsets of UV light sources: a primary set and a secondary set. During normal operation, only the primary subset of UV light sources is illuminated. If it is detected that the primary subset has failed, is inoperable or is otherwise not functioning adequately, the secondary subset of UV light sources is switched on. In other terms, the secondary subset of UV light sources is a backup or secondary set of light sources. It will be appreciated that alternative modes of operation may easily be envisaged within the scope of the present disclosure. In some examples, both the primary subset and the secondary subset of UV light sources are used during operation of the disinfection apparatus.

Further, each disinfection unit has a diagnostics element operable to determine whether the UV lights are functioning correctly and to provide information to an operator if there is a failure. In effect, if one light source fails, another will turn on and an error signal will be sent to the central control unit

The disinfection apparatus 100 comprises one or more positioning aids 124 operable to facilitate the correction positioning of the object to be disinfected. In an example, the positioning aids comprise at least one indent ridge positioned in the floor of the disinfection apparatus. The positioning aids may have any suitable or relevant shape or form. In the present example, the positioning aids are adapted to receive one or more wheels of a shopping trolley. When moved into the disinfection apparatus, the positioning aids facilitate the correct positioning of the shopping trolley. This ensures consistent operation of the disinfection apparatus. Further, speed of operation of the disinfection apparatus is increased, since users spend less time correctly placing the shopping trolley within the disinfection apparatus.

The disinfection apparatus has at least one proximity sensor 126 mounted inside the disinfection compartment 108.

In some examples, the proximity sensor is formed in a contact pad mounted on an inside surface on either or both of the actuating doors 104. When the shopping trolley is positioned inside the disinfection compartment, the user pushes until the shopping trolley comes into contact with the contact pad or is immediately proximal thereto. This causes the proximity sensor to detect the presence of the shopping trolley and allows the disinfection process to proceed.

In the present example, the proximity sensor 126 comprises a microwave motion sensor and two photoelectric sensors. The microwave motion sensor is positioned on a top inside surface of the disinfection compartment. This enables the microwave motion sensor to detect motion in substantially all of the disinfection compartment. The two photoelectric sensors are positioned on an inside side surface of the disinfection compartment, so as to detect the presence of the shopping trolley. Each of the photoelectric sensors comprise a radiation source and detector, as well as a reflective element.

In addition to detecting the presence of the object to be disinfected, at least one of the sensors comprised in the proximity sensor 126 is operable to detect whether a person or animal is present inside the disinfection compartment. Effectively, the proximity sensor performs the function of an internal obstruction sensor as described above.

It will be appreciated that the proximity sensor 126 described in the present example is exemplary, and that additional or alternative positioning sensors may be envisaged within the scope of the present disclosure. In some examples, alternative or additional sensors may be fitted so as to ensure correct operation of the apparatus. It will further be appreciated that the position of the proximity sensor, or one or more of the microwave motion sensor and two photoelectric sensors, is purely exemplary. Numerous specific and advantageous positions may be envisaged within the scope of the present disclosure.

In an example, at least one of the indent ridges comprises a pressure sensor that detects whether a shopping trolley wheel is positioned in the correct spot. Any suitable pressure sensor may be used, including (but by no means limited to) load cell sensors; or inductive pressure sensors.

The disinfection apparatus has at least one access ramp 128 positioned adjacent to the at least one actuating doors 104. The access ramp facilitates access to the disinfection compartment, in particular for wheeled objects such as shopping trolleys. The access ramp is fitted with a rubberized surface 130, which reduces the risk of persons or objects slipping thereon. In an example, the access ramp comprises a suitable pressure sensor operable to detect the presence of an object or person on the access ramp. Any suitable pressure sensor may be used, including (but by no means limited to) load cell sensors; or inductive pressure sensors.

The disinfection apparatus has guard rails 132 positioned adjacent to the actuating doors and the access ramp. The guard rails facilitate access to the disinfection compartment by aiding in correct alignment of the shopping trolley during entry and exit from the disinfection compartment.

An external obstruction sensor 134 is mounted on the end portion 102D above the actuating door 104 proximal to the guard rails 132. The obstruction sensor is operable to detect whether an object or a person is present within a first safety zone. In the present example, the first safety zone substantially comprises the area between the guard rails. By ensuring that no persons or objects are present within the first safety zone, the risk of interference with the operation of the disinfection apparatus is reduced. Additionally, the risk of any accidental exposure of a person to the disinfection operation, in case of the apparatus being improperly sealed, is reduced.

Additionally, in some examples, the obstruction sensor 134 is operable to detect the presence of an object or a user within the first safety zone prior to a disinfection operation. If an object or user is detected within the first safety zone, a signal is sent to the door control unit, so as to open the actuating door in order to allow an object to be inserted into the disinfection compartment.

In the present example, the obstruction sensor 134 comprises an infrared sensor mounted on the actuating door 104 proximal to the guard rails 132. The infrared sensor is operable to detect the presence of persons or animals inside the first safety zone. It will be appreciated that alternative or additional sensors or types of sensors could be envisaged by the skilled person within the scope of the present disclosure. Examples include, without limitation: microwave sensors; or pressure sensors.

The disinfection apparatus, in some examples, comprises additional features which may increase the efficacy of the operation of the apparatus or increase the safety of operation of the apparatus.

In the present example, the disinfection apparatus has at least one emergency trigger 136 which, when operated, causes the operation of the disinfection apparatus to be instantly interrupted. The at least one emergency trigger is located within the disinfection compartment. This enables a person who is accidentally trapped inside the disinfection compartment to prevent the disinfection operation from being carried out whilst they are inside. This feature functions in conjunction with the above-mentioned proximity sensor 126 operable to also detect movement of persons or animals inside the disinfection compartment. If either the emergency trigger or the proximity sensor is activated, the operation of the disinfection apparatus will be halted, switching off the disinfection units 118 and switching the actuating doors 104 to an open and unlocked position.

Prior to use, the disinfection apparatus is configured in a deployed state. In this state, the at least one access ramps is positioned and locked in an open state. Further, the disinfection apparatus is connected to a power source, such as a mains source. For transportation to and from a deployment location, the disinfection apparatus may be configurable into a transportation state.

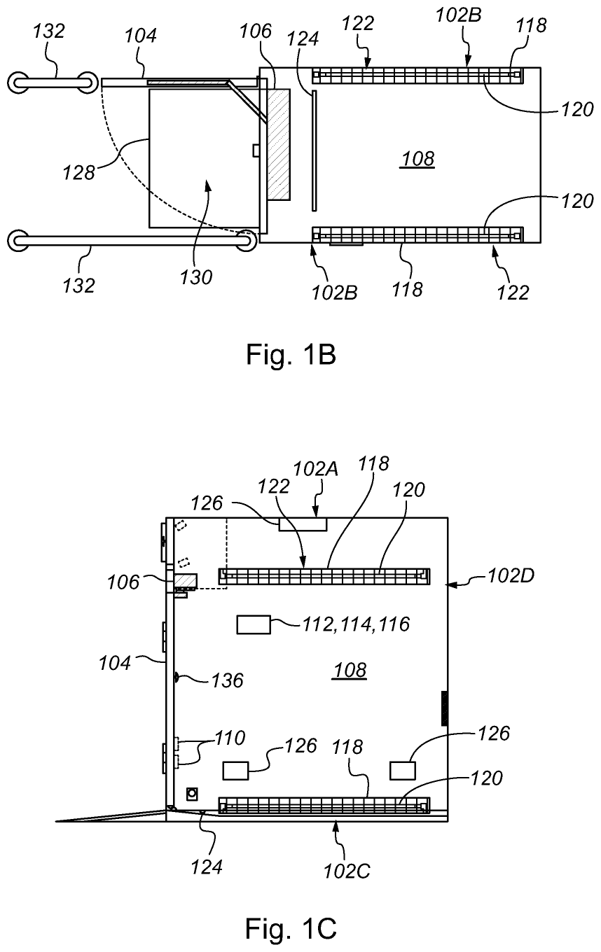

An example of use of the exemplary disinfection apparatus will now be described with reference to FIG. 2. It will be appreciated that this is purely for exemplary purposes and that further specific implementations may be envisaged by the skilled person.

In order to initiate a disinfection process, a user positions an object to be disinfected in a first location in the disinfection apparatus. Purely for exemplary purposes, the object to be disinfected is in the present example a shopping trolley. Accordingly, the terms object to be disinfected and shopping trolley will be used interchangeably in the following.

It will nevertheless be appreciated that the exemplary method as described in the following is equally applicable to other types of objects and in other situations or circumstances. Examples of such objects and circumstances include, without limitation: tools or hand tools; clothing; groceries; food stuffs; cooking utensils; food preparation utensils and equipment; medical instruments and personal protective equipment; electrical components such as mobile phones; jewellery; books and documentation; spectacles; glassware and crockery; potted plants; waste or refuse; toys; bed linen; cosmetics; baggage; or towels.

In some examples, in an optional method step 201A, prior to a positioning step, it is detected that the object to be disinfected is positioned in a second location. In some examples, the second location is external to the disinfection apparatus. In an example, the second location is substantially identical to the first safety zone. In a specific example, the second location is substantially between a pair of guard rails. Upon detecting the object in the second location, an actuating door is moved from a closed position into an open position to allow access to a disinfection compartment. In some examples, there is a time delay between detecting the object in the second location and the actuating door being actuated.

During the positioning step, the shopping trolley is positioned into a first location. In the present example, the first location is inside the disinfection compartment. In some examples, the first location is located substantially within the second safety zone described with reference to FIG. 1. The disinfection apparatus comprises one or more positioning aids to facilitate the positioning step. As described above, the one or more positioning aids may have any suitable shape or form in order to facilitate the correct positioning of the object to be disinfected. In the above-mentioned example, the one or more positioning aids comprises one or more indent ridges, each of said indent ridges being adapted to receive at least one shopping trolley wheel.

In a first method step 201, it is detected that the object is positioned in a first location within the disinfection apparatus. The detection may be carried out in any suitable fashion.

In an example, the step of detecting that the object to be disinfected is positioned in a first location comprises using a position sensor to detect that the object to be disinfected is positioned in the first location. The position sensor is, in some examples, positioned inside the disinfection compartment. As described above, in the present example, the at least one position sensor is a proximity sensor.

In a second method step 202, it is detected that at least a first safety zone is unobstructed. The detection step may be carried out in a suitable fashion. In some examples, the detection step is performed using one or more obstruction sensors. The one or more obstruction sensors are used to detect whether the at least one first safety zone is occupied by a person or object.

In some examples, the second method step further comprises detecting that the second safety zone only contains the object to be disinfected, the second safety zone being located inside a disinfection compartment. In such examples, the second method step further comprises using one or more suitable sensors to detect whether a second safety zone is occupied by a person or object. In the present example, wherein the disinfection apparatus is substantially identical to that described with reference to FIG. 1, this step is performed by the proximity sensor. As persons or animals generally move, as long as movement is detected it may safely be assumed that a person or animal is inside the disinfection compartment.

If a person or object is present within the at least one first safety zone or the presence of a person or animal is detected inside the second safety zone, any further method steps are prevented from being carried out. In some examples, the detection step is repeated or continuously performed until it is successfully detected that the one or more obstruction sensors are not triggered and the first safety zone is clear. In some examples, the method is interrupted until it is detected that the first safety zone is unobstructed. In specific examples, the method is interrupted for a predetermined interruption time period, after which the second method step is carried out again. The interruption time period may have any suitable length, such as (without limitation): 1 second; 2 seconds; 3 seconds; 5 seconds; or 10 seconds. It will be appreciated that these examples are purely for illustrative purposes, and that several variations of the second method step may be envisaged within the scope of the present disclosure.

In other examples, the method is interrupted and reset, thereby requiring the method to be restarted.

Once the second method step is successfully completed, the method proceeds to the next method step.

In a third method step 203, the disinfection apparatus is closed and locked. In the present example, one or more of the actuating doors is actuated and moved from an open position to a closed position. Subsequently, the one or more locking mechanisms corresponding to the respective actuating doors is engaged.

In some examples, the third method steps comprises a verification sub-step in which it is verified that the one or more actuating doors is correctly in the closed position. Additionally, the verification sub-step verifies whether the locking mechanisms are correctly engaged and in a locked configuration.

In a fourth method step 204, one or more disinfection operations is performed. The disinfection operation may be carried out in any suitable fashion using any suitable disinfection unit or units. In an example, the disinfection operation is carried out using the disinfection unit of the disinfection apparatus described in the above example.

In some examples, the disinfection operation comprises a plurality of disinfection steps or procedures.

In the present example, the disinfection operation comprises illuminating the object to be disinfected with radiation from the disinfection units comprised in the outer casing of the disinfection apparatus for a first disinfection period. The first disinfection period may be any suitable period of time, including (without limitation): 5 seconds; 10 seconds; 15 seconds; 20 seconds; 25 seconds; 30 seconds; 35 seconds; 40 seconds; 50 seconds; 60 seconds; 70 seconds; 80 seconds; or 90 seconds. In the present example, the first disinfection period is substantially 20 seconds. It will be appreciated that the specific duration is determined by the treatment, the specific type of bacteria or virus to be remove, and by the complexity and type of materials to be disinfected.

In a fifth method step 204, the disinfection apparatus is unlocked and opened. The fifth method step is substantially identical to the third method step, but carried out substantially in reverse.

In a sixth method step 206, it is detected that the object has been removed from the first location in the disinfection apparatus. The disinfection apparatus is then reset in order to render it ready for the next disinfection operation.

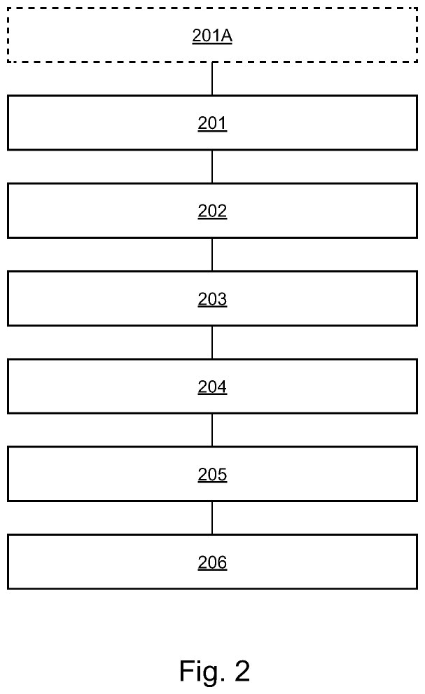

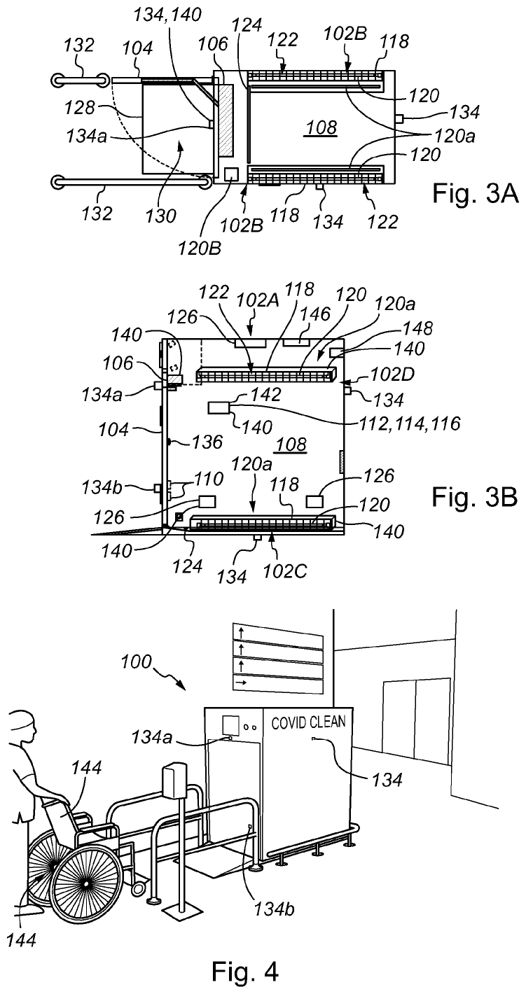

FIGS. 3A, 3B and 4 show another embodiment of the invention, which is substantially identical to the preceding embodiments with the following modifications.

As shown in FIG. 4, the disinfection apparatus 100 comprises a display unit 115 located on the actuating door 104. This provides a visual aid to the user, and indicates when the disinfection apparatus 100 is available to use. In other embodiments, the display unit 115 could be integrated in the actuating door 104.

In the embodiment shown in FIGS. 3A, 3B and 4, the disinfection apparatus 100 includes condition monitoring capability. The disinfection apparatus 100 comprises one or more condition monitoring devices operable to provide the status of each actuating door 104, the proximity sensor 126, the external obstruction sensor 134, and the at least one disinfection unit 118. The central control unit 114 is operable to obtain a status of each actuating door 104, the proximity sensor 126, the external obstruction sensor 134, and the disinfection unit 118 (including the light sources 120). The status includes an indication of whether the component is in compliance with one or more pre-defined operating parameters. The status includes the status of at least some of the components of each actuating door 104, the proximity sensor 126, the external obstruction sensor 134, and the disinfection unit 118 (including the light sources 120). Typically, the status includes one or more fault conditions.

One or more of the condition monitoring devices 140 are configured to provide a status of the light sources 120, and to measure a parameter associated with the operation of the light sources 120. In the embodiment shown in FIGS. 3A and 3B, the disinfection unit 118 comprises one or more backup light sources 120a (an example of a backup disinfection element). The central control unit 114 is operable to activate the backup light sources 120a in response to the status of the one or more light sources 120 being out of compliance with a predetermined value.

The display unit 115 is configured to indicate the status of each actuating door 104, the proximity sensor 126, the external obstruction sensor 134, and the disinfection unit 118 (including the light sources 120) and the one or more backup light sources 120a.

The central control unit 114 is operable to transmit one or more signals indicative of the status of each actuating door 104, the proximity sensor 126, the external obstruction sensor 134, and the disinfection unit 118 (including the light sources 120) to a computing device (not shown), a distributed computer network, and/or a computing device connected to a distributed computer network. The computing device could be a PC, laptop, or smart phone, or the like.

The central control unit 114 is operable to indicate one or more recommended scheduled maintenance periods, such as a service date, or the like, on the display unit 115.

The central control unit 114 is operable to store operation data associated with the use of the disinfection apparatus 100. The operation data includes the number of times the disinfection apparatus 100 was used, with a single use of the apparatus 100 being defined in any suitable way according to user preference, such as by one or more disinfection operations being carried out. The operation data includes the number of objects that have been disinfected using the disinfection apparatus 100. The operation data includes time data associated with how long the disinfection apparatus 100 has been used for, the time at which the disinfection apparatus 100 was used at and the date on which it was used.

The display unit 115 is operable to display at least some of the operation data, including the time data, the number of times the disinfection apparatus is used, and/or the number of objects that have been disinfected using the disinfection apparatus.

The central control unit 114 comprises an object communication element 142 operable to communicate with one or more identifier elements 144 associated with the object to be disinfected. The one or more identifier elements 144 are located in the object to be disinfected, although they could be located on, or adjacent to the object. The one or more identifier elements 144 are transceiver elements, although any suitable identifier element could be used, such as tags, RFID tags, internet-of-things (IoT) devices, or the like.

The object communication element 142 is operable to communicate with the one or more identifier elements 144 using one or more communication protocols. The communication protocol is WiFi, although any suitable protocol could be used.

The object communication element 142 is operable to obtain object data from the one or more identifier elements 144. The object data includes data on the type of object, the identity of the object (e.g. a unique identifier or code allocated to the object), information on when the object was disinfected by a disinfection apparatus, and location data associated with the object.

The central control unit 114 is operable to communicate the object data to one or more computing devices, optionally over a distributed computing network or wireless communication link.

The central control unit 114 is operable to display at least some of the object data on the display unit 115.

The disinfection apparatus 100 comprises an object sensor 146 operable to detect at least one property of the object to be disinfected when the object is located within the disinfection compartment 108. The at least one property includes the type of object. The object sensor 146 is operable to determine the shape of at least a portion of the object to the disinfected. The central control unit 114 is operable to classify the object according to the at least one property detected by the object sensor 146. The central control unit 114 is operable to classify the object according to the shape detected by the object sensor 146. The central control unit 114 is operable to classify the object as a simple shape object or a complex shape object.

The object sensor 114 is an optical sensor. However, in other embodiments, the, or each, object sensor could be an optical sensor, a camera, electro-optical sensor, infra-red sensor, or the like.

The central control unit 114 is operable to adjust the operating parameters of the at least one disinfection unit 118 based, at least in part, on data received from the object sensor 146. The central control unit 114 is operable to adjust the operating parameters of the light sources 120 based, at least in part, on data received from the object sensor 146. The central control unit 114 is operable to adjust the operating parameters of the disinfection unit 118 based, at least in part, on the classification of the object. The central control unit 114 is operable to adjust the operating parameters of the light sources 120 based, at least in part, on the classification of the object.

The disinfection apparatus 100 includes a camera 148 (an example component of a monitoring system), which is operable to record one or more images and/or one or more videos of at least a portion of the disinfection compartment 108 during disinfection operations.

The camera 148 is operable to detect shadows on the object to be disinfected during disinfection operations. The central control unit 114 is operable to modify the operation of the light sources 120 based on the detection of one or more shadows on the object to be disinfected by the camera 148. Modifying the operation of the light sources 120 includes activating or deactivating some or all of the light sources 120 and modifying the intensity of some or all of the light sources 120.

In some embodiments, modifying the operation of the light sources 120 can include activating or deactivating light sources 120 located at or adjacent to the inner top surface of the disinfection unit 118, the inner bottom surface of the disinfection unit 118 and/or at least one of the inner side surfaces of the disinfection unit 118, and/or modifying the intensity of the light sources 120.

One of the light sources 120b (shown in FIG. 3A) is movable between at least two positions in the disinfection unit 118. The movable light source 120b is mounted to a robotic vehicle configured to move the light source 120b between the at least two positions. The central control unit 114 is operable to control the operation of the actuator device. It will be understood that modifying the operation of the light sources 120 may include moving the movable light source 120b between the at least two positions.

The central control unit 114 is operable to trigger one or more alarms based on data received from the one or more external obstruction sensors 134. The one or more alarms can include visual alarms or warnings and/or audio alarms or warnings. The, or each, alarm may be displayed on the display unit 115.

The central control unit 114 is operable to prompt a person to use the disinfection apparatus 100. The central control unit 114 is operable to prompt a person to use the disinfection apparatus 100 based on data received from the one or more external obstruction sensors 134. The prompt may include a visual prompt and/or audio prompt. The, or each, prompt may be displayed on the display unit 115.

The external obstruction sensor 134 is operable to measure or infer a person's body temperature. The central control unit 114 is operable to trigger a warning or alarm if a person's body temperature is above a predetermined threshold and/or below a predetermined threshold.

The central control unit 114 is configured to automatically move the front actuating door 104 from a closed position to an open position in response to the detection of a person outside of the disinfection apparatus 100 by the one or more external obstruction sensors 134. In this embodiment, the user can use the apparatus 100 without providing any input to the apparatus, which is advantageous to mitigate the spread of infectious diseases, for cleanliness, and for ease of use.

The central control unit 114 is operable to move the front actuating door from the closed position to the open position when one or more of the external obstruction 134 sensors detects the presence of a user. The central control unit 114 is operable to move the front door 104 from the open position to the closed position when the object to be disinfected is in the first safety zone. The central control unit 114 is operable to move the rear actuating door 104 from the closed position to the open position when the one or more disinfection operations are complete. The complete operation of the apparatus 100 can therefore be performed without the user touching the apparatus 100.

In some embodiments, the central control unit 114 is configured to use data from the one or more external obstruction sensors 134 to classify the person detected by the one or more external obstruction sensors 134. For example, the person may be classified by height, weight, or any suitable parameter. The classification may include classifying the person as an adult or child, or an animal. The central control unit 114 is operable to allocate user permission to one or more classifications of a person and to deny user permission to one or more classifications of a person. The central control unit 114 is operable to maintain the at least one actuating door 104 in the closed position, if a person who does not have user permission is detected by the one or more external obstruction sensor 134. The central control unit 114 is operable to allow the operation of the disinfection apparatus 100 when a user who has user permission is detected by the one or more external obstruction sensors 134.

As shown in FIG. 3B, the disinfection apparatus 100 comprises a first external obstruction sensor 134a, orientated towards a first sensing area, and one or more second external obstructions sensors 134b, orientated towards a second sensing area. The first sensing area is at a substantially higher height than the second sensing area. There may be some overlap between the first and second sensing areas, or there may be substantially no overlap. The first sensing area may be defined to detect the presence of a first classification of a person at a predetermined distance range to the first external obstruction sensor 134a. The second sensing area may be defined to detect the presence of a second classification of a person at a predetermined distance range to the second external obstruction sensor 134b.

The second sensing area is defined to detect the presence of a child at a predetermined distance range to the second external obstruction sensor 134b and the first sensing area is defined to detect the presence of an adult at a predetermined distance range to the first external obstruction sensor 134a. The first sensing area is defined such that a child would not be detected at a predetermined distance range to the first external obstruction sensor 134a. In this example, the presence of an adult or child can be determined. It will be understood that the definition of adult and child can be set according to user preference and regulatory requirements. For example, the orientation of the sensors 134a, 134b could be used to determine a height threshold between the sensors, or sensor data could be used to infer whether an adult or child is present. The central control unit 114 is operable to deny user permission unless the first and second external obstruction sensors 134a, 134b both detect the presence of a person.

The central control unit 114 is operable to obtain a status of the position of the object in the disinfection compartment 108, and this status is indicated on the display unit 115 using a green status for the object being positioned correctly, and red for the object not being in the correct position.

The descriptions above are intended to be illustrative, not limiting. Thus, it will be apparent to one skilled in the art that modifications may be made to the invention as described without departing from the scope of the claims set out below.

For example, although not shown here, the monitoring system could comprise one or more UVA illumination sources operable to illuminate at least a portion of the object to be disinfected when the object is located in the disinfection unit. In this example, the monitoring system is operable to capture one or more images of the disinfection unit when illuminated, and to save the captured images. In this example, the monitoring system is configured to detect the presence of pathogens, bacteria or viruses. The detection of pathogens is carried out using fluorescence imaging, or any suitable imaging method of detecting or inferring the presence of pathogens, bacteria or viruses. The central control unit 114 could be operable to activate the one or more illumination sources of the monitoring system before a disinfection operation begins and/or after the disinfection operation is complete, to assess the success of the disinfection operation.

The disinfection apparatus could comprise an air supply system operable to provide a positive pressure difference between the inside of the disinfection unit and the outside of the disinfection unit. The air supply system could comprise one or more air inlets and one or more air outlets. The air inlet may be located at an upper region of the disinfection unit. In this arrangement, the pressure difference can mitigate the ingress of debris into the disinfection unit. This arrangement is also thought to assist in disinfecting the disinfection unit.

The disinfection unit could comprise one or more windows. The, or each, window may be configured to filter UV light. The, or each window may include one or more acrylic or plexiglass panes.

The disinfection apparatus could comprise one or more motors and one or more wheels, such that the disinfection apparatus can be moved. This is advantageous in some settings, such as a hospital setting.

The at least one actuating door could include an automatic elevating door mechanism, which may raise the door vertically.

In some embodiments, the disinfection apparatus may not comprise actuating doors. For example, the outer casing may be movable between an open and closed state, such as by raising the casing vertically, to allow an object to enter the disinfection unit, and lowering the casing to perform a disinfection operation (an elevator-type arrangement). It will be appreciated that moving the casing between an open and closed state can be carried out in a number of ways.

The disinfection apparatus could comprise an extension mechanism operable to configure the disinfection apparatus between a stowed state and a deployed state. The extension mechanism could be configured to extend the disinfection unit vertically and/or horizontally.

1-59. (canceled)

60. A disinfection apparatus comprising:
at least one disinfection unit operable to perform a disinfection operation on an object to be disinfected;
an outer casing;
at least one actuating door, the outer casing and the at least one actuating door defining a disinfection compartment;
at least one position sensor for detecting the presence of an object to be disinfected having been positioned within the disinfection compartment;
at least one obstruction sensor operable to detect whether at least a first safety zone is clear; and
a central control unit, the central control unit being operable to control operation of the at least one actuating door; the at least one position sensor; the at least one obstruction sensor; and the at least one disinfection unit.

61. The disinfection apparatus according to claim 60, wherein the at least one disinfection unit comprises a plurality of ultraviolet light sources operable to illuminate the object to be disinfected.

62. The disinfection apparatus according to claim 60, wherein:
the first safety zone is located externally to the outer casing; and
the at least one obstruction sensor comprises an external obstruction sensor operable to detect whether the first safety zone is clear.

63. The disinfection apparatus according to claim 60, further comprising:
a second safety zone, the second safety zone being substantially located inside the disinfection compartment; and
an internal obstruction sensor operable to detect whether the second safety zone is clear.

64. The disinfection apparatus according to claim 60, wherein the disinfection unit comprises one or more disinfection elements.

65. The disinfection apparatus according to claim 64, wherein:
the disinfection unit comprises one or more backup disinfection elements; and
the central control unit is operable to activate the one or more backup disinfection elements in response to the status of the one or more disinfection elements being out of compliance with a predetermined value.

66. The disinfection apparatus according to claim 60, wherein the central control unit is operable to transmit one or more signals indicative of the status of at least one of the at least one actuating door, the at least one position sensor, the at least one obstruction sensor, and the at least one disinfection unit, to at least one of computing device, a distributed computer network, and a computing device connected to a distributed computer network.

67. The disinfection apparatus according to claim 60, wherein:
the central control unit is operable to store operation data associated with the use of the disinfection apparatus; and
the operation data includes at least one of the number of times the disinfection apparatus was used, the number of objects that have been disinfected using the disinfection apparatus, time data associated with the how long the disinfection apparatus has been used for, the time at which the disinfection apparatus was used, and the date on which it was used.

68. The disinfection apparatus according to claim 60, wherein:
the central control unit comprises an object communication element;
the object communication element is operable to communicate with one or more identifier elements associated with the object to be disinfected;
the object communication element is operable to obtain object data from the one or more identifier elements; and
the object data includes data on at least one of the type of object, the identity of the object, information on when the object was disinfected by a disinfection apparatus, and location data associated with the object.

69. The disinfection apparatus according to claim 60, wherein the disinfection apparatus comprises one or more object sensors operable to detect at least one property of the object to be disinfected.

70. The disinfection apparatus according to claim 69, wherein:
the one or more object sensors are operable to detect at least one property of the object to be disinfected when the object is located within the disinfection compartment; and
the at least one property includes at least one of the type of object and the shape of at least a portion of the object to be disinfected.

71. The disinfection apparatus according to claim 70, wherein:
the central control unit is operable to classify the object according to the at least one property detected by the one or more object sensors;
the central control unit is operable to classify the object according to the shape detected by the one or more object sensors; and
the central control unit is operable to classify the object as a simple shape object or a complex shape object.

72. The disinfection apparatus according to claim 71, wherein the central control unit is operable to adjust the operating parameters of the at least one disinfection unit based, at least in part, on data received from the object sensor.

73. The disinfection apparatus according to claim 60, wherein:
the disinfection apparatus comprises a monitoring system operable to record one or more images and/or one or more videos of at least a portion of the disinfection compartment during disinfection operations; and
the monitoring system is operable to detect one or more shadows on the object to be disinfected.

74. The disinfection apparatus according to claim 73, wherein the central control unit is operable to modify the operation of the one or more disinfection elements based on the detection of one or more shadows on the object to be disinfected.

75. The disinfection apparatus according to claim 74, wherein:
one or more of the disinfection elements is movable between at least two positions in the disinfection unit; and
modifying the operation of the one or more disinfection elements includes moving one or more disinfection elements between the at least two positions.

76. The disinfection apparatus according to claim 60, wherein:
the central control unit is configured to use data from the one or more external obstruction sensors to classify a person detected by the one or more external obstruction sensors; and
the central control unit is operable to allocate user permission to one or more classifications of a person and to deny user permission to one or more classifications of a person.

77. The disinfection apparatus according to claim 76, wherein:
the disinfection apparatus comprises one or more first external obstruction sensors, orientated towards a first sensing area, and one or more second external obstructions sensors, orientated towards a second area;
the first sensing area is at a substantially higher height than the second sensing area;
the first sensing area is defined to detect the presence of a first classification of a person at a predetermined distance range to the first external obstruction sensor; and
the second sensing area is defined to detect the presence of a second classification of a person at a predetermined distance range to the second external obstruction sensor.

78. A method for controlling a disinfection apparatus for disinfecting an object to be disinfected, the method comprising:
detecting that the object to be disinfected is positioned in a first location within the disinfection apparatus;
detecting that at least a first safety zone is unobstructed;
closing and locking the disinfection apparatus;
performing one or more disinfection operations;
unlocking and opening the disinfection apparatus; and
resetting the disinfection apparatus when it is detected that object to be disinfected has been removed from the first location.

79. The method according to claim 78, wherein the step of detecting that at least a first safety zone is unobstructed comprises using one or more obstruction sensors to detect whether a second safety zone is occupied by a person or object, the second safety zone being located inside a disinfection compartment.
